SEQUENCE LISTING (7 sequences)

SEQ ID NO: 1 - protein, 15 aa, Yersinia pestis
VAHSVIGFIQRMFSE

SEQ ID NO: 2 - DNA, 45 bp, Yersinia pestis
gtggcccactctgtgattgggtttatccaacgcatgttctcggag

SEQ ID NO: 3 - protein, 9 aa, Yersinia pestis
SVIGFIQRM

SEQ ID NO: 4 - DNA, 27 bp, Yersinia pestis
tctgtgattgggtttatccaacgcatg

SEQ ID NO: 5 - protein, 219 aa, Yersinia pestis
MKISSFISTSLPLPTSVSGSSSVGEMSGRSVSQQTSDQYANNLAGRTESPQGSSLASRIIERLSSVAHSVIGFIQRMFSEGSHKPVVTPAPTPAQMPSPTSFSDSIKQLAAETLPKYMQQLNSLDAEMLQKNHDQFATGSGPLRGSITQCQGLMQFCGGELQAEASAILNTPVCGIPFSQWGTIGGAASAYVASGVDLTQAANEIKGLAQQMQKLLSLM

SEQ ID NO: 6 - DNA, 660 bp, Yersinia pestis
atgaaaatatcatcatttatttctacatcactgcccctgccgacatctgtgtcaggatctagcagcgtaggagaaatgtctgggcgctcagtctcacagcaaacaagtgatcaatatgcaaacaatctggccgggcgcactgaaagccctcagggttccagcttagccagccgtatcattgagaggttatcatcagtggcccactctgtgattgggtttatccaacgcatgttctcggaggggagccataaaccggtggtgacaccagcacccacacctgcacaaatgccaagtcctacgtctttcagtgacagtatcaagcaacttgctgctgagacgctgccaaaatacatgcagcagttgaatagcttggatgcagagatgctgcagaaaaatcatgatcagttcgctacgggcagcggccctcttcgtggcagtatcactcaatgccaagggctgatgcagttttgtggtggggaattgcaagctgaggccagtgccatcttaaacacgcctgtttgtggtattcccttctcgcagtggggaactattggtggggcggccagcgcgtacgtcgccagtggcgttgatctaacgcaggcagcaaatgagatcaaagggctggcgcaacagatgcagaaattactgtcattgatgtga

SEQ ID NO: 7 - protein, 8 aa, Artificial Sequence
  Synthetic
SIINFEKL

(19) United States
(12) Patent Application Publication
(10) Pub. No.: US 2013/0273092 A1
(43) Pub. Date: Oct. 17, 2013

(54) USES OF YERSINIA YOPE PEPTIDE, GENE AND SUBPARTS THEREOF AS A PLAGUE VACCINE COMPONENT AND ASSAYS FOR YERSINIA PESTIS-SPECIFIC T CELLS

(71) Applicants: Stephen T. Smiley, Saranac Lake, NY (US); Jr-shiuan Lin, Saranac Lake, NY (US)

(72) Inventors: Stephen T. Smiley, Saranac Lake, NY (US); Jr-shiuan Lin, Saranac Lake, NY (US)

(73) Assignee: Trudeau Institute, Sarananc Lake, NY (US)

(21) Appl. No.: 13/879,334

(22) PCT Filed: Oct. 20, 2011
(86) PCT No.: PCT/US11/57033

§ 371 Date: Jun. 14, 2013

Related U.S. Application Data
(60) Provisional application No. 61/405,986, filed on Oct. 22, 2010.

(51) Int. Cl. A61K 39/02

(52) U.S. Cl. CPC A61K 39/0291
USPC 424/190.1; 424/234.1; 435/7.24

(57) ABSTRACT

This invention relates to vaccine formulations comprising the Yersinia pestis YopE peptide antigen or subparts thereof. The invention also relates to methods of vaccinating subjects at risk of Yersinia pestis infection as well as assays for measuring immune response to plague vaccines.

### STATEMENT OF GOVERNMENT SUPPORT

This invention was made with government support under Grant No. RO1A161577 awarded by the National Institutes Of Health. The government has certain rights in the invention.

### FIELD OF THE INVENTION

The present invention relates generally to a vaccine comprising at least a portion of the amino acid sequence encoding the YopE peptide, at least a portion of the nucleic acid sequence encoding the YopE peptide, or combinations thereof. In some embodiments this invention relates to a method of vaccinating a subject at risk of being infected by a pathogen by administering a composition comprising at least a portion of the amino acid sequence encoding the YopE peptide, at least a portion of the nucleic acid sequence encoding the YopE peptide, or combinations thereof to the subject. In other embodiments, this invention relates to a method of monitoring an immune response by reacting (i.e. inoculating) a mammal with a plague vaccine to elicit an immune response and reacting the cells produced by that immune response with a YopE peptide such that cells generated by the immune response are identified by their binding or response to the peptide.

### BACKGROUND

Plague is an infectious disease that affects animals and humans. It is caused by the bacterium Yersinia pestis (Y. pestis), which is found in rodents and their fleas. Plague occurs in many areas of the world, including the United States. Although easily destroyed by sunlight and drying, Y. pestis can survive for up to one hour when aerosolized. Plague infections may manifest in several forms, including bubonic, pneumonic and septicemic plague. Depending on the circumstances these forms may occur separately or in combination. Bubonic plague, the classic and most common form of plague, typically occurs following a bite from an infected flea or introduction of Y. pestis contaminated material through a rupture of the skin. Patients develop swollen and tender lymph glands (buboes), fever, headache, chills and weakness. Bubonic plague does not spread from person to person. Pneumonic plague occurs when Y. pestis infects the lungs of an individual. This form of infection permits person-to-person spread of aerosolized bacteria and usually requires direct and close contact with an infected person or animal. Pneumonic plague may also occur if a person with bubonic or septicemic plague is not treated and the bacteria spread to the lungs. Pneumonic plague also may occur if Y. pestis is purposefully aerosolized, for example as a result of an act of bioterrorism. Septicemic plague occurs when plague bacteria multiply in the blood. Such infections may occur independently or result from complications following pneumonic or bubonic plague. Infected individuals experience fever, chills, abdominal pain, shock, and bleeding into skin and other organs.

Individuals in direct contact with infected patients typically undergo prophylactic antibiotic treatment for 7 days. Antibiotics such as streptomycin, gentamicin, the tetracyclines, sulfonamides and chloramphenicol are effective against pneumonic plague and should be administered within 24 hours of the first symptoms to reduce the likelihood of death.

Two types of plague vaccine are currently used in various parts of the world—a live vaccine derived from an attenuated Y. pestis strain and a killed, formalin-fixed vaccine derived from a virulent Y. pestis strain. These vaccines are typically administered only to individuals considered to be at high risk for Y. pestis infection, including individuals who work with or are potentially exposed to fully virulent strains and military personnel serving in areas where plague is endemic. Although these vaccines indicate the feasibility of protecting against disease, they have a number of shortcomings. The live attenuated vaccine is highly reactogenic and is not licensed for use in humans in the United States or Europe. The killed whole cell vaccine, also reactogenic, requires multiple doses and is no longer licensed for use in the United States. Antibodies elicited by the killed vaccine wane relatively quickly, requiring booster inoculations every 1 to 2 years. Additionally, experimental evidence indicates that the killed whole cell plague vaccine does not provide protection against the pneumonic form of the disease.

What is needed is a vaccine that protects against Yersinia pestis infection as well as an assay to measure immune responses following immunization with such a vaccine.

### SUMMARY OF THE INVENTION

The present invention relates generally to a vaccine formulation comprising the Y. pestis YopE peptide antigen or subparts thereof. In some embodiments the YopE peptide antigen comprises the amino acid sequence of SEQ ID NO: 1. In other embodiments the subpart of the YopE peptide antigen comprises the amino acid sequence of SEQ ID NO: 3. In other embodiments the YopE peptide antigen comprises the amino acid sequence of SEQ ID NO: 5 or subparts thereof. In one embodiment the YopE peptide antigen comprises the N-terminal portion of the amino acid sequence of SEQ ID NO: 5 (e.g. the first 20 amino acids). In one embodiment the YopE peptide antigen comprises the C-terminal portion of the amino acid sequence of SEQ ID NO: 5 (e.g. the last 20 amino acids). In one embodiment the YopE peptide antigen comprises the internal portion of the amino acid sequence of SEQ ID NO: 5 (e.g. the second 20 amino acids, the third 20 amino acids, the fourth 20 amino acids, the fifth 20 amino acids, the sixth 20 amino acids, the seventh 20 amino acids, the eighth 20 amino acids or the ninth 20 amino acids). In further embodiments the formulation further comprises a pharmacologically acceptable buffer, an excipient and/or an adjuvant. In still further embodiments the formulation comprises additional antigens.

In some embodiments the invention relates to a vaccine formulation comprising a nucleic acid sequence encoding the Y. pestis YopE peptide antigen or subparts thereof. In some embodiments the nucleic acid sequence is in a vector. In other embodiments the nucleic acid sequence comprises the nucleotide sequence of SEQ ID NO: 2. In other embodiments the nucleic acid sequence comprises the nucleotide sequence of SEQ ID NO: 6 or subparts thereof. In one embodiment the nucleotide sequence comprises the 5′ portion of the nucleotide sequence of SEQ ID NO: 6 (e.g. the first 60 nucleotides). In one embodiment the nucleotide sequence comprises the 3′ portion of the nucleotide sequence of SEQ ID NO: 6 (e.g. the last 60 nucleotides). In one embodiment the nucleotide sequence comprises the internal portion of the nucleotide sequence of SEQ ID NO: 6 (e.g. the second 60 nucleotides, the third 60 nucleotides, the fourth 60 nucleotides, the fifth 60 nucleotides, the sixth 60 nucleotides, the seventh 60 nucleotides, the eighth 60 nucleotides or the ninth 60 nucleotides). In other embodiments the nucleic acid sequence comprises the nucleotide sequence of SEQ ID NO: 4. In further embodiments the formulation further comprises a pharmacologically acceptable buffer, an excipient and/or an adjuvant. In still further embodiments the formulation comprises additional antigens.

In some embodiments the invention relates generally to a method of vaccination comprising, providing: a subject at risk of being infected by Y. pestis and a Y. pestis YopE peptide antigen or subpart thereof, and a administering the peptide or subpart thereof to the subject. In some embodiments an immune response against the YopE peptide antigen is induced in the subject. In other embodiments the YopE peptide antigen comprises the amino acid sequence of SEQ ID NO: 1. In other embodiments the YopE peptide antigen subpart comprises the amino acid sequence of SEQ ID NO: 3. In other embodiments the YopE peptide antigen comprises the amino acid sequence of SEQ ID NO: 5, or subparts thereof. In one embodiment the YopE peptide antigen comprises the N-terminal portion of the amino acid sequence of SEQ ID NO: 5 (e.g. the first 20 amino acids). In one embodiment the YopE peptide antigen comprises the C-terminal portion of the amino acid sequence of SEQ ID NO: 5 (e.g. the last 20 amino acids). In one embodiment the YopE peptide antigen comprises the internal portion of the amino acid sequence of SEQ ID NO: 5 (e.g. the second 20 amino acids, the third 20 amino acids, the fourth 20 amino acids, the fifth 20 amino acids, the sixth 20 amino acids, the seventh 20 amino acids, the eighth 20 amino acids or the ninth 20 amino acids). In other embodiments immune response is a cell-mediated immune response. In further embodiments the cell-mediate immune response is a T cell response. In further embodiments the T cell response is a CD8 T cell response. In still further embodiments the immune response provides protective immunity against Y. pestis in the subject. In some embodiments the administration further comprises a pharmacologically acceptable buffer, an excipient and/or an adjuvant. In some embodiments the YopE peptide antigen is administered to the subject along with other antigenic peptides. In some embodiments the YopE peptide is expressed by a vector. In other embodiments the YopE peptide is administered to the subject by the intramuscular, intravenous, inhalation and/or subcutaneous routes.

In some embodiments the invention relates generally to a method of vaccination comprising, providing: a subject at risk of being infected by Y. pestis and a vector comprising a nucleic acid sequence encoding the Y. pestis YopE peptide or subpart thereof, and administering the nucleic acid sequence to the subject. In some embodiments the YopE peptide encoded by the nucleic acid is expressed in the subject such that an immune response against the YopE peptide is induced. In other embodiments the nucleic acid comprises the nucleotide sequence of SEQ ID NO: 2. In other embodiments the nucleic acid comprises the nucleotide sequence of SEQ ID NO: 6 or subparts thereof. In one embodiment the nucleotide sequence comprises the 5′ portion of the nucleotide sequence of SEQ ID NO: 6 (e.g. the first 60 nucleotides). In one embodiment the nucleotide sequence comprises the 3′ portion of the nucleotide sequence of SEQ ID NO: 6 (e.g. the last 60 nucleotides). In one embodiment the nucleotide sequence comprises the internal portion of the nucleotide sequence of SEQ ID NO: 6 (e.g. the second 60 nucleotides, the third 60 nucleotides, the fourth 60 nucleotides, the fifth 60 nucleotides, the sixth 60 nucleotides, the seventh 60 nucleotides, the eighth 60 nucleotides or the ninth 60 nucleotides). In other embodiments the nucleic acid comprises the nucleotide sequence of SEQ ID NO: 4. In other embodiments the immune response is a cell-mediated immune response. In further embodiments the cell-mediated immune response is a T cell response. In further embodiments the T cell response is a CD8 T cell response. In still further embodiments the immune response provides protective immunity against Y. pestis in the subject. In some embodiments the vector is administered to the subject along with a second vector encoding additional antigenic peptides on the same or other vectors. In other embodiments the vector is a plasmid, cosmid, fosmid, BAC, YAC, bacterial expression vector (for example Salmonella) or viral expression vector (for example adenovirus). In other embodiments the vector is administered to the subject by intramuscular, intravenous, inhalation and/or subcutaneous routes. In some embodiments the invention relates generally to a method of vaccination comprising, providing: a subject at risk of being infected by Y. pestis, a Y. pestis YopE peptide antigen or subpart thereof, and a dendritic cell (or other antigen presenting cell) from the subject; exposing (i.e. pulsing) the dendritic cell ex vivo with the YopE peptide antigen or subpart thereof, and transferring the dendritic cell back to the subject. In some embodiments an immune response against the YopE peptide antigen is induced in the subject. In other embodiments the YopE peptide antigen comprises the amino acid sequence of SEQ ID NO: 1. In other embodiments the YopE peptide antigen subpart comprises the amino acid sequence of SEQ ID NO: 3. In other embodiments the YopE peptide antigen comprises the amino acid sequence of SEQ ID NO: 5 or subparts thereof. In one embodiment the YopE peptide antigen comprises the N-terminal portion of the amino acid sequence of SEQ ID NO: 5 (e.g. the first 20 amino acids). In one embodiment the YopE peptide antigen comprises the C-terminal portion of the amino acid sequence of SEQ ID NO: 5 (e.g. the last 20 amino acids). In one embodiment the YopE peptide antigen comprises the internal portion of the amino acid sequence of SEQ ID NO: 5 (e.g. the second 20 amino acids, the third 20 amino acids, the fourth 20 amino acids, the fifth 20 amino acids, the sixth 20 amino acids, the seventh 20 amino acids, the eighth 20 amino acids or the ninth 20 amino acids). In other embodiments immune response is a cell-mediated immune response. In further embodiments the cell-mediate immune response is a T cell response. In further embodiments the T cell response is a CD8 T cell response. In still further embodiments the immune response provides protective immunity against Y. pestis in the subject. In some embodiments the administration further comprises a pharmacologically acceptable buffer, an excipient and/or an adjuvant. In some embodiments the dendritic cell is transferred to the subject along with other antigenic peptides. In other embodiments the dendritic cell is transferred to the subject by the intramuscular, intravenous, inhalation and/or subcutaneous routes. In some embodiments the invention relates generally to an assay comprising, providing: a cell and a MHC-I tetramer/YopE peptide complex, reacting the MHC-I tetramer/YopE peptide complex with the cell under conditions that permit a receptor on the cell to bind to the MHC-I tetramer/YopE peptide complex, and detecting the cell bound to the complex. In some embodiments the cell is an immune cell. In other embodiments the immune cell is a T cell. In other embodiments the T cell is a CD8 T cell. In further embodiments the receptor is a T cell receptor. In still further embodiments the cell is from a subject infected with Y. pestis. In some embodiments the cell is from a subject immunized with a vaccine against Y. pestis. In some embodiments the peptide is a Y. pestis YopE peptide or subpart thereof. In other embodiments the YopE peptide comprises the amino acid sequence of SEQ ID NO: 1. In other embodiments the YopE peptide comprises the amino acid sequence of SEQ ID NO: 3. In other embodiments the YopE peptide comprises the amino acid sequence of SEQ ID NO: 5 or subparts thereof. In one embodiment the YopE peptide antigen comprises the N-terminal portion of the amino acid sequence of SEQ ID NO: 5 (e.g. the first 20 amino acids). In one embodiment the YopE peptide antigen comprises the C-terminal portion of the amino acid sequence of SEQ ID NO: 5 (e.g. the last 20 amino acids). In one embodiment the YopE peptide antigen comprises the internal portion of the amino acid sequence of SEQ ID NO: 5 (e.g. the second 20 amino acids, the third 20 amino acids, the fourth 20 amino acids, the fifth 20 amino acids, the sixth 20 amino acids, the seventh 20 amino acids, the eighth 20 amino acids or the ninth 20 amino acids). In still other embodiments the MHC-I tetramer is fluorescently labeled. In some embodiments the cell to bound to the MHC-I tetramer/peptide complex is detected by flow cytometry.

In further embodiments, YopE may serve as a useful component of assays measuring the CD8 T cell responses to plague vaccines, whether those responses are induced by immunization with YopE, subparts of YopE, genes encoding YopE or its subparts; mixtures of YopE or its subparts with other antigens; or live agents expressing YopE or its subparts. In some embodiments, YopE peptides may serve as components in ELISA, ELISpot, and MHC multimer based assays. In other embodiments, the YopE peptides may be used to measure the responses of YopE-specific CD8 T cell in mice expressing the K^{b} MHC molecule. In further embodiments, these and/or other YopE peptides may be useful for other animals, including humans, expressing distinct MHC molecules.

### DEFINITIONS

To facilitate the understanding of this invention a number of terms are defined below. Terms defined herein (unless otherwise specified) have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, terms defined in the singular are intended to include those terms defined in the plural and vice versa.

As used herein, the terms “patient” and “subject” refer to a human or animal who is ill or who is undergoing treatment for disease, but does not necessarily need to be hospitalized. For example, out-patients and persons in nursing homes are “patients”.

As used herein the phrase, “subject at risk for plague” refer to subjects who have an increased probability of being exposed and/or infected with Y. pestis. Subjects at risk for plague generally have one or more risk factors for infection, including but not limited to, living in areas characterized by overcrowding, poor sanitation and/or high rodent populations. Subjects exposed to susceptible animals, including for example, rodents, wild rodents, rabbits, squirrels, prairie dogs, cats and the fleas associated with such animals are considered to have an elevated risk of being infected by Y. pestis. Since Y. pestis represents a potential agent for bioterrorism, in one embodiment a “subject at risk for plague” may also include military personnel and the general population.

As used herein, the terms “peptide”, “peptide sequence”, “amino acid sequence”, “polypeptide”, and “polypeptide sequence” are used interchangeably herein to refer to at least two amino acids or amino acid analogs that are covalently linked by a peptide bond or an analog of a peptide bond. The term peptide includes oligomers and polymers of amino acids or amino acid analogs. The term peptide also includes molecules that are commonly referred to as peptides, which generally contain from about two (2) to about twenty (20) amino acids. The term peptide also includes molecules that are commonly referred to as polypeptides, which generally contain from about twenty (20) to about fifty amino acids (50). The term peptide also includes molecules that are commonly referred to as proteins, which generally contain from about fifty (50) to about three thousand (3000) amino acids. The amino acids of the peptide may be L-amino acids or D-amino acids. A peptide, polypeptide or protein may be synthetic, recombinant or naturally occurring. A synthetic peptide is a peptide that is produced by artificial means in vitro.

As used herein, the term “portion” or “subpart” when used in reference to a protein (as in “a portion of a given protein”) refers to fragments of that protein. The fragments may range in size from four amino acid residues to the entire amino sequence minus one amino acid.

As used herein, the term “antibody” or “antibodies” refers to globular proteins (“immunoglobulins”) produced by cells of the immune system to identify and neutralize foreign antigens. “Monoclonal antibodies” (mAb) are antibodies that recognize a specific antigenic epitope (i.e. monospecific) because they are derived from clones of a single hybridoma. Hybridomas are cells engineered to produce a desired mAb antibody in large amounts. Briefly, B-cells are removed from the spleen of an animal that has been challenged with the desired antigen. These B-cells are then fused with myeloma tumor cells that can grow indefinitely (i.e. immortal) in culture. Since the fused cell or hybridoma is also immortal it will multiply rapidly and indefinitely to produce large amounts of the desired mAb (Winter and Milstein, Nature, 349, 293-299, 1991). “Polyclonal antibodies” (pAb) are a mixture of antibodies that recognize multiple epitopes of a specific antigen. Polyclonal antibodies are produced by immunizing an animal (i.e. mouse, rabbit, goat, horse, sheep etc.) with a desired antigen to induce B-lymphocytes to produce antibodies to multiple epitopes of that antigen. These antibodies can then be isolated from the animal's blood using well-known methods, such as column chromatography.

As used herein, the terms “reduce” and “reduction” and grammatical equivalents thereof, means lowering, decreasing, or diminishing in degree, intensity, extent, and/or amount. As used herein, it is not necessary that there be complete reduction, it is sufficient for there to be some reduction.

As used herein, the terms “prevent”, “preventing” and grammatical equivalents thereof, indicates the hindrance of the recurrence, spread or onset of a disease or disorder. It is not intended that the present invention be limited to complete prevention. In some embodiments, the onset is delayed, or the severity of the disease or disorder is reduced.

As used herein, the terms “treat”, “treating”, “treatment” and grammatical equivalents thereof, refers to combating a disease or disorder, as for example in the management and care of a patient. “Treatment” is not limited to cases where the subject (e.g. patient) is cured and the disease is eradicated. Rather, the present invention also contemplates treatment that merely reduces symptoms, improves (to some degree) and/or delays disease progression. It is not intended that the present invention be limited to instances wherein a disease or affliction is cured. It is sufficient that symptoms are reduced.

As used herein, a “diagnostic” is a compound or method that assists in the identification and characterization of a health or disease state. With regard to the present invention, it is contemplated that a method for measuring specific CD8 T cell response to a plague vaccine can be used as a diagnostic to evaluate the course of an immune response in a person following inoculation with said vaccine and/or following infection with a Y. pestis vaccine. For example, a patient inoculated with a live attenuated Y. pestis vaccine may be examined with such a diagnostic to determine whether a particular cellular (i.e. CD8 T cell) and/or cytokine response (i.e. TNF-α and IFN-γ) has been stimulated as well as the relative levels of such cells and cytokines.

As used herein, the term “cytokines” refers to a category of protein, peptide, or glycoprotein molecules secreted by specific cells of the immune system that carry signals between cells. Cytokines are a critical component of both the innate and adaptive immune response, and are often secreted by immune cells that have encountered a pathogen to activate and recruit additional immune cells to increase the system's response to the pathogen. Cytokines are typically released in the general region (i.e. vicinity) of the pathogen such that responding immune cells arrive at that site of infection and become activated. In one embodiment the responding immune cells include phagocytes (i.e. phagocytic cells) that ingest and destroy pathogens (including bacteria). In one embodiment the pathogen may be free-floating (i.e. not attached to a cell), cell-associated (i.e. on the surface of a cell) and/or associated with cellular fragments (i.e. cellular debris) resulting from lysed or ruptured cells. In another embodiment, the pathogen may reside within cells. Each individual cytokine has at least one matching cell-surface receptor; in some instances an individual cytokine may have multiple matching cell-surface receptors. Upon binding of a cytokine to its cell-surface receptor a cascade of intracellular signaling events alters the cell's function. This includes the upregulation and/or downregulation of genes involved in the production of other cytokines, an increase expression of surface receptors for other molecules, or suppression of the cytokine itself by feedback inhibition. The effect of a particular cytokine on a given cell depends on the cytokine, its extracellular abundance, the presence and abundance of the complementary receptor on the cell surface, and downstream signals activated by receptor binding. Common cytokines include interleukins that are responsible for communication between white blood cells; chemokines that promote chemotaxis; and interferons that have anti-viral effects, such as shutting down protein synthesis in the host cell. Cytokines are characterized by considerable “redundancy”, in that many cytokines appear to share similar functions. Tumor necrosis factor alpha (TNF-α) is a cytokine involved in systemic inflammation and is a member of a group of cytokines that stimulate the acute phase reaction. A primary role of TNF-α is in the regulation of immune cells. TNF-α is also able to induce apoptotic cell death, induce inflammation and inhibit tumorigenesis and viral replication. TNF-α is produced by T cells, macrophages and a broad variety of other cell types including lymphoid cells, mast cells, endothelial cells, cardiac myocytes, adipose tissue, fibroblasts, and neuronal tissue. Large amounts of TNF-α are released in response to bacterial products including lipopolysaccharide (LPS) and Interleukin-1 (IL-1). Interferon-gamma (IFN-γ) is a cytokine that is critical for innate and adaptive immunity against viral and bacterial infections as well as tumor control. Aberrant IFN-γ expression is associated with a number of auto-inflammatory and autoimmune diseases. IFN-γ is secreted by a number of cell types including macrophages and NK cells as part of the innate immune response, and by CD4 and CD8 T cells once antigen-specific immunity develops.

As used herein, the term “lymphocyte” refers to white blood cells that include B lymphocytes (B cells) and T lymphocytes (T cells). Individual B cells and T cells may specifically recognize a single antigenic epitope, although in some instances they are more promiscuous and are capable of specifically recognizing multiple antigenic epitopes. Individual T and B cells also recognize the body's own (self) tissues as different from non-self tissues. In one embodiment, the inability of T and B cells to differentiate between “self” and “non-self tissues” is referred to as “autoimmunity”. After B cells and T cells are activated, a sub-population will multiply and provide “memory” for the immune system. This allows the immune system to respond faster and more efficiently the next time the individual is exposed to the same antigen.

As used herein, the term “CD8 T cell” refers to a T cell sub-group that express CD8 glycoprotein molecules on their surface. Typically, CD8 T cells are capable of inducing the death of infected somatic or tumor cells, cells that are infected with pathogens (such as viruses or bacteria) or cells that are otherwise damaged or dysfunctional. In addition to the CD8 glycoprotein, CD8 T cells express T-cell receptors (TCRs) that recognize a specific antigenic peptide bound to polymorphic major histocompatibility complex class I (MHC-I) molecules present on all nucleated cells. In one embodiment, the CD8 TCR recognizes a specific antigenic peptide (for example, YopE peptide) presented by (i.e. bound to) an MHC-I molecule by binding to the MHC-I/peptide complex, rather than the antigenic peptide alone. In one embodiment, CD8 T cells identify cells infected by Y. pestis, or tissues of the body infected by Y. pestis, due to the presence of specific antigens (for example, YopE) presented by MHC-I molecules. These CD8 cells then become activated and secrete cytokines (such as TNF-α and IFN-γ) in the vicinity of the infected cells and tissues. These cytokines then activate and recruit phagocytic cells and/or other lymphocytes to that region to eliminate the infection. The phagocytes ingest bacteria that are free-floating (i.e. not attached to a cell), cell-associated (i.e. on the surface of a cell) and/or associated with cellular fragments (i.e. cellular debris) resulting from lysed or ruptured cells. In some instances, the bacteria are intracellular (i.e. residing within cells) and the CD8 T cells lyse the infected cells, or cause widespread destruction of the infected tissue, thereby eliminating the bacteria directly and/or helping the phagocytes to eliminate the bacteria.

As used herein, the term “cell-mediated immunity” refers to an immune response that does not necessarily require antibodies or complement but rather involves the activation of macrophages, natural killer cells (NK), antigen-specific T cells and the release of various cytokines in response to an antigen. Patterns of cytokine production by T cells are associated with different immunological responses, including type-1 (Th-1) and type-2 (Th-2) responses. In some embodiments, the Th-1 response stimulates cell-mediated immunity by releasing cytokines such as IFN-γ, which increase the production of IL-12 by dendritic cells (DCs) and macrophages. In some embodiments, IL-12 also stimulates the production of IFN-γ in Th-1 cells by positive feedback.

As used herein, the term “vaccine”, “vaccinate” or “vaccination” refers to the introduction of a small amount of an antigen into an organism in order to trigger the immune system to activate B cells and/or sensitize T cells. These cells recognize the antigen and also establish immune system “memory” such that future exposures to the antigen result in its rapid recognition. A variety of antigenic substances may be used for vaccination, including dead, inactivated or live attenuated organisms (including bacteria) or purified products derived therefrom. Vaccines can be used to prevent or ameliorate the effects of a future infection (i.e. prophylactic) or they can be used to treat existent infections or conditions, such as cancer (i.e. therapeutic).

As used herein, the term “MHC multimer based assay” refers to a method of detecting, monitoring and/or isolating antigen-specific T cells using soluble major MHC-I/peptide tetramers. In one embodiment, the assay employs four biotinylated MHC-I molecules, which individually consist of a tripartite complex of heavy chain domains (alpha1, alpha2, and alpha3), light chain domains (beta-2-microglobulin) and a peptide antigen. Attaching the biotinylated MHC-I molecules to streptavidin provides an MHC-I/peptide tetramer complex with four stable and specific binding sites for the T cell receptors on CD8 T cells. The streptavidin molecule may be labeled (for example, fluorescently labeled) such that the tetramer bound with antigen-specific CD8 T cells may be detected (for example, by flow cytometry). In one embodiment, this methodology allows for the rapid quantification of antigen-specific T cells.

As used herein, the term “ELISA” or “Enzyme-linked immunosorbent assay” refers to a biochemical technique used to detect the presence of a specific compound or target molecule in a sample. ELISAs are often used in immunology to detect the presence of a specific antibody, antigen or cytokine in a sample. In one embodiment, a cytokine-specific antibody is affixed to a surface, and a culture supernatant is washed over the surface so that cytokine present within the supernatant can bind to the cytokine-specific antibody. Then, a second cytokine-specific antibody is washed over the surface. This second antibody is linked to an enzyme or molecule capable of providing a detectable signal. Such a signal may be fluorescence such that exposure to light of the appropriate wavelength allows bound cytokine/antibody complexes to fluoresce. The amount of cytokine in the sample may then be determined based on the magnitude of the fluorescence.

As used herein, the term “ELISPOT assay” or “Enzyme-Linked Immunosorbent Spot Assay” refers to a method for monitoring immune responses in humans and animals developed by Cecil Czerkinsky. The ELISPOT assay is a modified version of the ELISA immunoassay and was originally developed to enumerate B cells secreting antigen-specific antibodies. This assay has subsequently been adapted for various tasks, including the identification and enumeration of cytokine-producing CD8 T cells at the single cell level. Briefly, the ELISPOT assay permits visualization of the secretory product of individual activated or responding cells. Each “spot” that develops in the assay represents a single reactive cell. Thus, the ELISPOT assay provides both qualitative (type of antigen (e.g. YopE) being recognized) and quantitative (number of responding cells) information. The sensitivity of the ELISPOT assay permits frequency analysis of rare cell populations (e.g., YopE-specific memory responses). This sensitivity is due in part to the ability to rapidly capture the product around the secreting cell before it is diluted in the supernatant, captured by receptors of adjacent cells, or degraded. Limits of detection are below 1/100,000 rendering the assay uniquely useful for monitoring antigen-specific responses, applicable to a wide range of areas of immunology research, including cancer, transplantation, infectious disease, and vaccine development.

As used herein, the term “intramuscular” refers to a mode of administration of a substance such as a drug or vaccine by directly introducing the compound within the substance of a muscle.

As used herein, the term “intravenous” refers a mode of administration of a substance such as a drug or vaccine, within or into a vein.

As used herein, the term “subcutaneous” refers to a mode of administration of a substance such as a drug or vaccine by introducing the compound beneath the skin (intradermally or subdermally) such that the body absorbs it.

As used herein, the term “inhalation” refers to a mode of administration of a substance such as a drug or vaccine by the nasal (i.e. intranasal) or oropharyngeal respiratory route for local or systemic effect by drawing an aerosol into the lungs with the breath.

As used herein, the term “immunization” refers to the process whereby a subject is made immune or resistant to an infectious disease by stimulating the immune system to protect against subsequent infection or disease. Exposure of a subject's immune system to foreign (i.e. non-self) molecules (i.e. antigens) allows the immune response to develop immunological memory such that subsequent encounters with that antigen may elicit a qualitatively and/or quantitatively altered response. Typically, the secondary response is faster and stronger. The swift response to subsequent encounters with a foreign molecule is due the production of memory B cells and memory T cells.

As used herein, the term “inoculation” refers to the introduction of a vaccine, antigen or other foreign molecule into the body of a subject.

As used herein, the term “challenge” refers to the administering of an antigen or other foreign substance to a subject to observe the physiological response that occurs. A “challenge” may include, for example, introducing a pathogen into a previously sensitized or immunized subject to evoke and assess the resulting immunologic response.

### BRIEF DESCRIPTION OF THE DRAWINGS

For a more complete understanding of the features and advantages of the present invention, reference is now made to the detailed description of the invention along with the accompanying figures.

FIG. 1 depicts the survival of wild type mice following intravenous injections of either diluent (DMEM), 5×10⁶ naïve splenocytes or 5×10⁶ cloned CD8 T cells (Vβ6+ or Vβ6−) prior to intranasal challenge with 2×10⁵ CFU of Y. pestis.

FIG. 2 depicts the proliferation and IFN-γ production of CD8 T cell clones (Vβ6+ or Vβ6−) grown at 26° C. or 37° C. and stimulated with syngeneic mitomycin c-treated splenocytes and bacterial strains that either contain the pCD1 plasmid (Y. pestis D27; Y. pestis C092) or lack the pCD1 plasmid (Y. pestis D28; and E. coli).

FIG. 3 depicts the IFN-γ production of a representative CD8 T cell clone stimulated with syngeneic mitomycin c-treated splenocytes and recombinant E. coli strains expressing individual pCD1-encoded proteins.

FIG. 4 depicts the proliferation and IFN-γ production of a representative CD8 T cell clone stimulated with syngeneic mitomycin c-treated splenocytes and individual 15-mer YopE peptides. Each 15-mer YopE peptide (42 total) overlaps its neighboring peptide(s) by 10 amino acids.

FIG. 5 depicts the activation (TNF-α and IFN-γ expression) of CD4 and CD8 T cells isolated from the lungs of mice immunized with attenuated Y. pestis and stimulated with control ovalbumin peptide, YopE peptide or anti-CD3 monoclonal antibody.

FIG. 6 depicts the survival rates of mice immunized with DCs pulsed with either YopE peptide or ovalbumin peptide (control).

FIG. 7 depicts the survival rates of mice immunized with cholera toxin adjuvant alone or cholera toxin mixed with YopE peptide.

FIG. 8 depicts the percentage of CD8 T cells (from the lung and peripheral blood) stained with an MHC K^{b} tetramer loaded with a YopE peptide from mice immunized with attenuated Y. pestis and then challenged with virulent Y. pestis (as compared to naïve mice or mice that were immunized but not challenged).

FIG. 9 depicts a schematic example of an MHC-I/peptide tetramer employed in the MHC multimer based assay.

### DETAILED DESCRIPTION OF THE INVENTION

The present invention relates generally to a vaccine comprising at least a portion of the amino acid sequence encoding the YopE peptide, at least a portion of the nucleic acid sequence encoding the YopE peptide, or combinations thereof. In some embodiments this invention relates to a method of vaccinating a subject at risk of being infected by a pathogen by administering a composition comprising at least a portion of the amino acid sequence encoding the YopE peptide, at least a portion of the nucleic acid sequence encoding the YopE peptide, or combinations thereof to the subject. In other embodiments, this invention relates to a method of monitoring an immune response by reacting (i.e. inoculating) a mammal with a plague vaccine to elicit an immune response and reacting the cells produced by that immune response with a YopE peptide such that cells generated by the immune response are identified by their binding or response to the peptide.

I. Yersinia pestis YopE

Yersinia pestis (Y. pestis) diverged recently from the enteric pathogen Yersinia pseudotuberculosis, differing in the presence of specific virulence plasmids but is not as closely related to Y. enterocolitica. Chromosomal DNAs from wild type Y. pestis and Y. pseudotuberculosis show a very high degree of relatedness. A comparison of the complete genomic sequence of Y. pseudotuberculosis IP32953 and a panel of Y. pestis isolates from around the world reveal 32 Y. pestis chromosomal genes that together with the two Y. pestis-specific plasmids likely represent the genetic material acquired by Y. pestis since its divergence from Y. pseudotuberculosis. In contrast, 317 genes and 149 other pseudogenes detected in Y. pseudotuberculosis were absent from Y. pestis, indicating that as many as 13% of Y. pseudotuberculosis genes no longer function in Y. pestis. It therefore appears that extensive insertion sequence-mediated genome rearrangements and reductive evolution through massive gene loss, resulting in the elimination and modification of pre-existing gene expression pathways, plays a larger role than gene acquisition in the evolution of Y. pestis. Despite their close genetic relationship, Y. pestis and Y. pseudotuberculosis differ radically in their pathogenicity and transmission, emphasizing the ability of a highly virulent epidemic clone to suddenly emerge from a less virulent, closely related progenitor (PNAS Sep. 21, 2004 (101) 38: 13826-13831).

Y. pestis is a Gram-negative rod-shaped bacterium belonging to the family Enterobacteriaceae. It is a facultative anaerobe that can infect humans and other animals. Human Y. pestis infection takes three main forms: pneumonic, septicemic and bubonic plagues. All three forms are widely believed to have been responsible for a number of high-mortality epidemics throughout human history. Pathogenesis due to Y. pestis infection of mammalian hosts is due to several factors including the ability to suppress and avoid normal immune system responses such as phagocytosis thereby allowing it to proliferate inside lymph nodes and cause lymphadenopathy. To this end Y. pestis, Y. pseudotuberculosis and Y. enterocolitica all host the 70-75 kb pCD1 plasmid. However, Y. pestis hosts two additional plasmids that are not carried by the other Yersinia species—the 9.5 kb pPCP1 plasmid (also referred to as pPla or pPst) and the 100-110 kb pMT1 plasmid (also referred to as pFra). These three plasmids encode several virulence proteins that direct the pathogenesis of Y. pestis. pFra encodes a phospholipase D that is important for Y. pestis to be transmitted by fleas, while pPla encodes a protease that activates plasminogen and is an important virulence factor for bubonic plague. Many of the Y. pestis virulence proteins are anti-phagocytic in nature, including roles in bacterial adhesion, invasion and injection of proteins into host cells.

a) Type III Secretion System

Many Gram-negative bacteria use a plasmid-encoded type III secretion system (T3SS) to translocate toxins into mammalian cells. Several important human pathogens, including Salmonella typhimurium, Pseudomonas aeruginosa, and multiple Yersinia species use the T3SS to translocate virulence factors into host cells. All three pathogenic Yersiniae species (Y. pestis, Y. pseudotuberculosis, Y. enterocolitica) encode a T3SS on the pCD1 virulence plasmid. This T3SS comprises more than 40 constituents; including transport substrates called Yersinia outer proteins (Yops), specific Yop chaperones (Sycs) and Yop secretion proteins (Ysc). The plasmid-encoded T3SS system allows extracellular Yersinia docked at the surface of cells or localized in phagosomes to inject Yop effector proteins into the cytosol of these cells. These effector Yops disturb the dynamics of the cytoskeleton, block phagocytosis by macrophages and polymorphonuclear leukocytes (PMNs) and impair the production of pro-inflammatory cytokines, chemokines and adhesion molecules. These actions allow the survival of the invading Yersinia and their multiplication in lymphoid tissues.

b) Yersinia Outer Proteins

As described above, Yersinia virulence is dependent on the expression of plasmid-encoded secreted proteins called Yops, which include YopA, YopB, YopD, YopE, YopH, YopJ YopM, YopO and YopT. Some of the Yops are transported into the host cell cytoplasm by the T3SS. Three of the Yops (YopB, YopD and LcrV) are thought to form pores in the host cell membrane through which YopA, YopE, YopH, YopJ, YopM, YopO and YopT are injected. The Yops then limit phagocytosis and cell signaling pathways. Four of these Yops (YopH, YopE, YopT and YpkA/YopO) contribute to the impairment of phagocytosis by disturbing the dynamics of the cytoskeleton; three of these (YopE, YopT and YpkA/YopO) act on monomeric GTPases, whereas YopH is a powerful protein tyrosine phosphatase that dephosphorylates focal adhesions and complexes that are involved in signaling adhesion. YopT is a cysteine protease that inhibits RhoA by removing the isoprenyl group that is important for localizing the protein to the cell membrane. It has been proposed that YopE and YopT may also function to limit YopB/D-induced cytolysis to prevent YopB/D-induced rupture of host cells and release of cell contents that would attract and stimulate immune system responses. In addition to its antiphagocytic role, YopH also inhibits lymphocyte proliferation and the synthesis of monocyte chemotactic protein 1 (MCP1). YopP downregulates the inflammatory response of macrophages, epithelial and endothelial cells by blocking the mitogen-activated protein kinase (MAPK) and nuclear factor-κB (NF-κB) pathways. YopM is a protein comprised of leucine-rich repeats that travels to the nucleus of the target cell. Its role and target, however, are still unknown. Loss of any of the Yop effectors generally leads to a marked decrease in the virulence of Yersinia in infected mice.

c) GTPase-Activating Proteins

Guanine nucleotide binding proteins (G proteins) are a family of proteins that function as molecular switches due to their ability to function differentially depending on the number of phosphate groups attached to the guanine nucleotides that they bind. They belong to the larger group of enzymes called GTPases, a family of hydrolase enzymes that can bind and hydrolyze guanosine triphosphate (GTP). When G proteins are attached to a complex with three phosphate groups (i.e. GTP) they are activated. When they are attached to a complex with only two phosphate groups (i.e. GDP) they are inactivated. Guanine nucleoside triphosphatase (GTPase)-activating proteins (GAPs) are a family of regulatory proteins that bind to activated G proteins to stimulate their GTPase activity such that the G proteins switch from the GTP-bound form (active) to the GDP-bound form (inactive). This hydrolysis can be reverted (i.e. switching the G protein on again) by guanine nucleotide exchange factors (GEFs). Only the active state of the G protein can transduce a signal to a reaction chain. The ability of GAPs to turn G proteins off depends on their activity state and local concentration in the cell. The GAPs that act on monomeric GTP-binding proteins of the Ras superfamily have conserved structures and use similar mechanisms, whereas most GAPs that act on alpha subunits of heterotrimeric G proteins belong to a distinct family—the regulator of protein signaling (RGS) protein family.

In Yersinia the Yop effectors that inhibit phagocytosis (YopE, YopT and YpkA/YopO) act on monomeric GTPases of the Rho family, a family of small G proteins (approximately 21 kDa) that is a subfamily of the Ras superfamily. Members of the Rho GTPase family have been shown to regulate many aspects of intracellular actin dynamics and are found in all eukaryotic organisms including yeasts and some plants. The GAP translocated by Yersinia, YopE, is critical for virulence. It disrupts actin filaments and suppresses ingestion of Yersinia by phagocytes.

The Rho-family of proteins are anchored to the inner side of the plasma membrane by a prenyl group covalently attached to the proteins' carboxy-terminal end. Rho, Rac and Cdc42 are members of the Rho family that are known to control the dynamics of the cytoskeleton. YopE acts as a GAP, switching RhoA, Rac and Cdc42 to their ‘off’ form by accelerating GTP hydrolysis. YopE has an arginine finger motif similar to those found in eukaryotic GAP proteins, and exchanging Arg144 from this motif with an alanine residue results in the loss of both GAP activity and the ability to induce cytotoxicity in cultured cells. The GAP activity of YopE is equally effective on RhoA, Rac and Cdc42 in vitro, but it is predominantly active on Rac in vivo, as shown in primary endothelial cells. This GAP activity gives YopE its antiphagocytic function.

### II. Infection

Plague is a zoonotic disease primarily affecting rodents, with transmission between rodents accomplished by their associated fleas. The “sylvatic cycle” (or “enzootic cycle”) refers to the portion of the Y. pestis transmission sequence spent cycling between wild animals and vectors. This is opposed to the “domestic” or “urban” cycle, in which Y. pestis cycles between vectors and non-wild, urban or domestic animals. In both the urban and sylvatic cycles most of the Y. pestis spreading occurs between rodents and fleas. Humans do not play a role in the long-term survival of Y. pestis and often serve as incidental or “dead-end” hosts. Humans may exhibit differing infection rates from these cycles due to transmission efficiencies and environmental exposure levels. Infected animals or insects, typically fleas, may transmit infections to humans through contact with skin tissue. Humans can also spread the bacteria to other humans through sneezing, coughing or direct contact with infected tissue.

a) Bubonic Plague

Bubonic plague is the classic form of the disease. Infection typically occurs when an infected flea bites a person or when Y. pestis contaminated materials enter through a break in the skin. Within 2 to 6 days of contact with Y. pestis patients typically develop symptoms of fever, headache, chills, weakness and extremely tender swollen lymph nodes (buboes). Gastrointestinal complaints such as nausea, vomiting, and diarrhea are also common. Skin lesions infrequently develop at the initial site of an infection. Depending on the site of the initial infection any of the lymph node areas can be involved, soreness in the affected lymph nodes will sometimes precede swelling. Buboes are typically found in the inguinal and femoral regions but also occur in other nodes. Bacteremia or secondary plague septicemia is frequently seen in patients with bubonic plague. Bubonic plague does not typically spread from person to person.

b) Pneumonic Plague

Primary pneumonic plague is a rare but deadly disease that occurs when Y. pestis infects the lungs via respiratory droplets through close contact with an infected individual. Unlike bubonic and septicemic plague, person-to-person (or animal) spread of pneumonic plague can occur by inhaling aerosolized Y. pestis suspended in respiratory droplets. Becoming infected in this way usually requires direct and close contact with the ill person or animal. Pneumonic plague may also occur if a person with bubonic or septicemic plague is untreated and the bacteria spread to the lungs. The incubation period for primary pneumonic plague is typically 1 and 3 days. Symptoms typically progress rapidly from a febrile flu-like illness to an overwhelming pneumonia with coughing and the production of bloody sputum. The first signs of illness usually include fever, headache, weakness, rapidly developing pneumonia with shortness of breath, chest pain, cough and sometimes bloody or watery sputum. The pneumonia progresses for 2 to 4 days and may cause respiratory failure and shock. Without early treatment, patients may die. From 1970 to 1993 about 2% of the plague cases in the United States were diagnosed as primary pneumonic plague, the vast majority of which were contracted from infected cats. The last case of pneumonic plague in the United States that was the result of person-to-person spread occurred during the 1924-1925 epidemic in Los Angeles.

c) Septicemic Plate

Primary septicemic plague occurs when plague bacteria multiply in the blood, with patients generally diagnosed by positive blood cultures with no palpable lymphadenopathy. Septicemic plague can occur by itself or arise as a complication of pneumonic or bubonic plague. When occurring alone septicemic plague is caused in the same ways as bubonic plague, however, without the development of buboes. Clinically, septicemic plague resembles septicemias caused by other gram-negative bacteria. Patients typically experience fever, chills, prostration, abdominal pain, shock and bleeding into skin and other organs. The mortality rate for people with septicemic plague ranges from 30% to 50%, due at least in part to the fact that antibiotics generally used to treat undifferentiated sepsis are not effective against Y. pestis. Septicemic plague does not spread from person to person. In the 30-year period between 1947 and 1977 about 10% of U.S. plague patients were diagnosed with primary septicemic plague. However, in the early 1980s in New Mexico, 25% of plague patients had primary septicemic plague.

### III. Disease Prevention

Both antibiotics and vaccines have been used to prevent Y. pestis infections. Antibiotics are typically administered only as prophylactic measures following close contact with a pneumonic plague patient. All forms of plague are treated with antibiotics, although some antibiotic-resistant strains have been reported. The most commonly used antibiotics are gentamicin, streptomycin, tetracycline, sulfonamides or chloramphenicol. Individuals who have been exposed to infected fleas or who have been within 2 meters of a coughing patient with pneumonic plague should be treated with antibiotics before they develop symptoms. To reduce the chance of death, antibiotics must be given within 24 hours of first symptoms. Antibiotic treatment for 7 days will generally protect individuals who have had direct, close contact with infected patients. Wearing a close-fitting surgical mask can protect close contacts from acquiring pneumonic infection.

Live attenuated and killed whole cell vaccines against disease caused by Y. pestis have been available since the early part of the last century. Although these vaccines indicate the feasibility of protecting against disease, they have a number of shortcomings. The most widely used live attenuated vaccine is derived from a pigmentation-negative strain related to EV76, however, it is highly reactogenic and is not licensed for use in humans in the United States. The killed whole cell vaccine is a formalin-fixed virulent strain of Y. pestis that is also reactogenic, provides poor protection against pneumonic plague and immunization requires multiple doses of the vaccine. A vaccine manufactured by Greer Laboratories (Lenoir, N.C.) from Y. pestis 195/P and administered intramuscularly as a series of three primary shots is no longer available. When still in use the initial dose comprised a 1.0 ml of a suspension containing 1.8×10⁹ to 2.2×10⁹ fixed bacteria/ml; this dose was followed 1 to 3 months later by a 0.2-ml dose. A third primary injection of 0.2 ml was given 5 to 6 months after the second dose. Two booster doses of 0.2 ml were administered at 6-month intervals, and additional booster shots were administered every 1 to 2 years. Only individuals considered to be at high risk for Y. pestis infection were administered the vaccine, including those who work with (or are potentially exposed to) fully virulent strains and military personnel serving in areas where plague is endemic. Evidence that the vaccine is effective against plague in humans was indirectly based on the number of confirmed plague cases in U.S. military personnel during World War II and in Vietnam.

There is a need for a new plague vaccine for a number of reasons. First, the vaccine described above (which is no longer available) caused an adverse reaction in a significant percentage of recipients. Although the reactions were generally mild, they could be severe. In addition, the antibodies induced by the vaccine waned quickly and therefore required booster inoculations every 1 to 2 years. Experimental evidence also indicated that the vaccine did not protect against the pneumonic form of the disease. A variety of potential vaccine candidates have been described, including rationally attenuated mutants, subunit vaccines and naked DNA vaccines. In one embodiment, an injected subunit vaccine may represent a near-term solution for a vaccine that protects against both bubonic and pneumonic plague (Expert Opinion on Biological Therapy, June 2004, (4) δ: 965-973).

### IV. Plague as a Weapon

Weaponization of Y. pestis is difficult since it is easily destroyed by drying, heat and ultraviolet light. However, when released into the air the bacterium may survive for up to one hour depending on conditions. During World War II the Japanese bred infected fleas by the billions and released them over northern Chinese cities causing numerous epidemics. Recently Y. pestis has gained attention as a possible biological warfare agent and the Centers for Disease Control and Prevention (CDC) has classified it as a category A pathogen requiring preparation for a possible terrorist attack.

V. Yersinia pseudotuberculosis

Y. pseudotuberculosis primarily causes disease in animals although humans are occasionally infected, most often through the food-borne route. In animals, Y. pseudotuberculosis can cause tuberculosis-like symptoms, including localized tissue necrosis and granulomas in the spleen, liver and lymph node. In humans, symptoms are similar to those of infection with Y. enterocolitica except that the diarrheal component is often absent, which sometimes makes the resulting condition difficult to diagnose. Y. pseudotuberculosis infections can mimic appendicitis, especially in children and younger adults, and, in rare cases the disease may cause skin complaints (erythema nodosum), joint stiffness and pain (reactive arthritis) or spread of bacteria to the blood (bacteremia). Infection usually becomes apparent 5-10 days after exposure and typically lasts 1-3 weeks without treatment. In complex cases or those involving immunocompromised patients, antibiotics may be necessary for resolution; ampicillin, aminoglycosides, tetracycline, chloramphenicol or a cephalosporin may all be effective. Although Y. pseudotuberculosis is usually only able to colonize hosts by peripheral routes and cause serious disease in immunocompromised individuals, if the bacterium gains access to the blood stream it has an LD50 comparable to Y. pestis at only 10 colony forming units (CFU).

VI. Yersinia enterocolitica

Yersiniosis is an infectious disease caused by bacteria of the genus Yersinia. Most human illness in the United States is caused by one species, Y. enterocolitica. While only a few strains of Y. enterocolitica cause illness in humans, infections that do occur may elicit a variety of symptoms depending on the age of the individual. Infection with Y. enterocolitica occurs most often in young children. Common symptoms in children are fever, abdominal pain and diarrhea, which is often bloody. Symptoms typically develop 4 to 7 days after exposure and may last 1 to 3 weeks or longer. In older children and adults right-sided abdominal pain and fever may be the predominant symptoms and may be confused with appendicitis. In a small proportion of cases complications such as skin rash, joint pain and/or spread of bacteria to the bloodstream can occur. The major animal reservoir for Y. enterocolitica strains that cause human illness is pigs, but Y. enterocolitica strains are also found in rodents, rabbits, sheep, cattle, horses, dogs and cats. In pigs, the bacteria are most likely to be found on the tonsils.

Y. enterocolitica infections are typically acquired by eating contaminated food, especially raw or undercooked pork products. The preparation of raw pork intestines (chitterlings) may be particularly risky. Infants can be infected if their caretakers handle raw chitterlings and then fail to adequately clean their hands before handling the infant or the infant's toys, bottles or pacifiers. Drinking contaminated unpasteurized milk or untreated water can also transmit the infection. Occasionally Y. enterocolitica infection occurs after contact with infected animals. On rare occasions it can be transmitted as a result of the bacterium passing from the stool or soiled fingers of one person to the mouth of another person. This may happen when basic hygiene and hand washing habits are inadequate. The organism is rarely transmitted through contaminated blood during a transfusion.

### EXPERIMENTAL

The following are examples that further illustrate embodiments contemplated by the present invention. It is not intended that these examples provide any limitations on the present invention.

In the experimental disclosure that follows, the following abbreviations apply: eq. or eqs. (equivalents); M (Molar); μM (micromolar); N (Normal); mol (moles); mmol (millimoles); μmol (micromoles); nmol (nanomoles); pmoles (picomoles); g (grams); mg (milligrams); μg (micrograms); ng (nanogram); vol (volume); w/v (weight to volume); v/v (volume to volume); L (liters); ml (milliliters); μl (microliters); cm (centimeters); mm (millimeters); μm (micrometers); nm (nanometers); C (degrees Centigrade); rpm (revolutions per minute); DNA (deoxyribonucleic acid); kDa (kilodaltons).

Results demonstrate that Y. pestis YopE is a dominant antigen recognized by CD8 T cells that protect against pneumonic plague in a mouse model. Following intranasal challenge with virulent Y. pestis, nearly all the anti-CD3-activatable CD8 T cells in the lung recognize YopE. These YopE-specific CD8 T cells simultaneously produce TNF-α and IFN-γ ex vivo in an intracellular cytokine assay, and provide mice with significant protection from lethal disease caused by pulmonary infection with virulent Y. pestis

I. CD8 T Cell Clones Derived from Mice that Survive Y. Pestis Infection Protect Against Virulent Y. Pestis Challenge

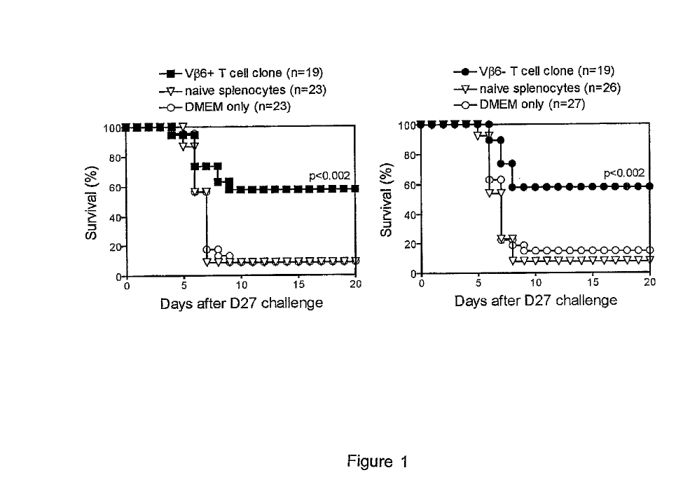

Results demonstrate that multiple CD8 T cell clones derived from mice that survived Y. pestis infection confer protection against lethal pulmonary challenge with virulent Y. pestis (FIG. 1). T cell clones were isolated from wild type C57BL/6 mice that had been immunized with attenuated Y. pestis strain D27pLpxL and then challenged 62 days and 92 days later with virulent Y. pestis strain D27. After another 35 days, mice were euthanized and splenocytes were harvested and re-stimulated in vitro in bulk culture with Y. pestis strain D27 as antigen. Two weeks later, the resulting T cell line was cloned at limiting dilution using syngeneic mitomycin c-treated splenocytes as antigen presenting cells and Y. pestis strain D27 as antigen. Clones were maintained by re-stimulating every 2 weeks using syngeneic mitomycin c-treated splenocytes as antigen presenting cells and Y. pestis strain D27 as antigen. FIG. 1 depicts the survival of wild type C57BL/6 mice that received intravenous injections of diluent (DMEM), 5×10⁶ naïve splenocytes, or 5×10⁶ of a Vb6+ CD8 T cell clone (left panel) or a Vb6− CD8 T cell clone (right panel) on the day prior to intranasal challenge with 2×10⁵ colony forming units of Y. pestis strain D27. Mice that received T cell clones showed significantly improved survival as compared with mice that received DMEM or naïve splenocytes (p<0.002 by log rank test; each graph depicts data pooled from 4 independent experiments).

II. CD8 T Cell Clones Derived from Mice that Survive Y. Pestis Infection Recognize An Antigen Encoded by, or Dependent Upon, the pCD1 Plasmid

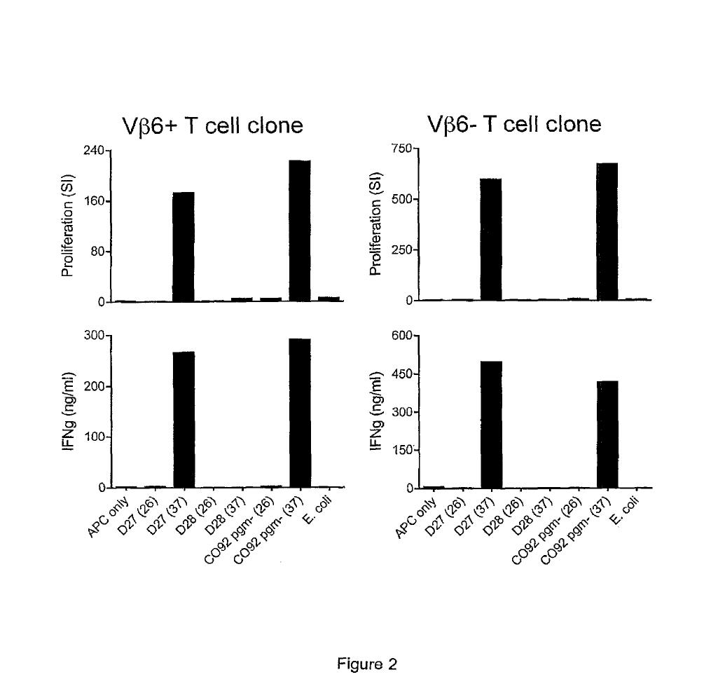

CD8 T cell clones derived from mice that survive Y. pestis infection recognize an antigen encoded by, or dependent upon, the pCD1 plasmid (FIG. 2). The specificity of the protective CD8 clone depicted in FIG. 1 was assessed in vitro by re-stimulating the clone with syngeneic mitomycin c-treated splenocytes as antigen presenting cells (APC) and the indicated bacterial strains as antigen. The strains were grown at either 26° C. or 37° C. as indicated. Strains included Y. pestis D27; Y. pestis D28, which lacks the pCD1 plasmid; Y. pestis strain C092, which represents a different biovar than D27, and E. coli. Culture supernatants were harvested 48 hr after initiation of culture and assayed for IFN-γ by ELISA. ³H-thymidine was added 48 hr after the initiation of culture. Incorporation of ³H-thymidine was measured at 72 hr and graphed as fold increase above APC only (Stimulation index, SI). Both the Vb6+CD8 T cell clone (left panels) and Vb6− CD8 T cell clone (right panels) produced IFN-γ and proliferated in response to pCD1-harboring bacteria grown at 37° C. pCD1-encoded Yop proteins are known to be induced under these conditions.

III. CD8 T Cell Clones Derived from Mice that Survive Y. Pestis Infection Recognize the Full Length YopE Protein

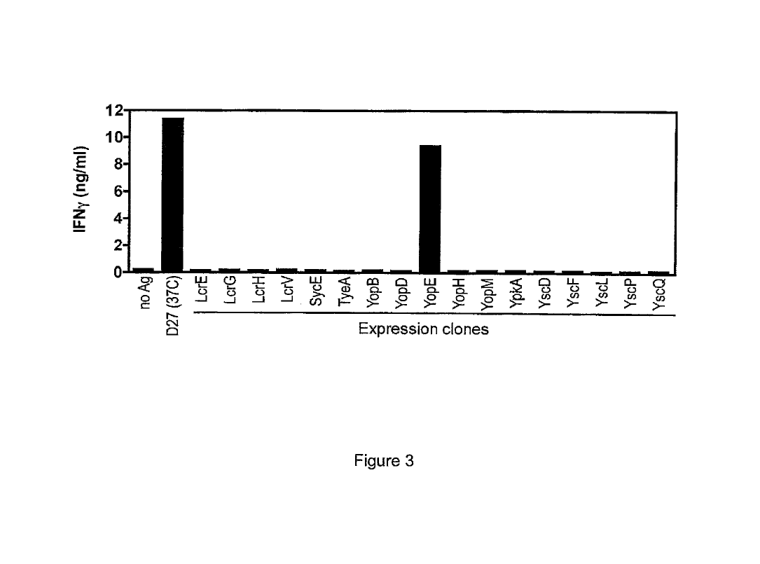

CD8 T cell clones derived from mice that survive Y. pestis infection recognize YopE (FIG. 3). The specificity of the protective CD8 clones depicted in FIGS. 1 and 2 was assessed in vitro by re-stimulating the clones with syngeneic mitomycin c-treated splenocytes as antigen presenting cells (APC) and the indicated bacterial strains as antigen. Specifically, the CD8 T cell clones were assayed for responsiveness to Y. pestis strain D27 and individual recombinant E. coli strains engineered to express the indicated pCD1-encoded proteins. The clones responded specifically to an E. coli strain expressing Y. pestis YopE (SEQ ID NO: 5 and SEQ ID NO: 6). IFN-γ production from a representative clone is depicted. Culture supernatants were harvested 48 hr after initiation of culture and assayed for IFN-γ by ELISA.

The amino acid sequence of the full length YopE peptide is H₂N-MKISSFISTSLP LPTSVSGSSSVGEMSGRSVSQQTSDQYANNLAGRTESPQGSSLASRIIERLSSV AHSVIGFIQRMFSEGSHKPVVTPAPTPAQMPSPTSFSDSEKQLAAETLPKYMQQ LNSLDAEMLQKNHDQFATGSGPLRGSITQCQGLMQFCGGELQAEASAILNTP VCGIPFSQWGTIGGAASAYVASGVDLTQAANEIKGLAQQMQKLLSLM-OH (SEQ ID NO: 5).

The nucleotide sequence encoding the amino acid of SEQ ID NO: 5 is 5′-ATGAAAATATCATCATTTATTTCTACATCACTGCCCCTGCCGACATCTGTG TCAGGATCTAGCAGCGTAGGAGAAATGTCTGGGCGCTCAGTCTCACAGCA AACAAGTGATCAATATGCAAACAATCTGGCCGGGCGCACTGAAAGCCCTC AGGGTTCCAGCTTAGCCAGCCGTATCATTGAGAGGTTATCATCAGTGGCC CACTCTGTGATTGGGTTTATCCAACGCATGTTCTCGGAGGGGAGCCATAA ACCGGTGGTGACACCAGCACCCACACCTGCACAAATGCCAAGTCCTACGT CTTTCAGTGACAGTATCAAGCAACTTGCTGCTGAGACGCTGCCAAAATAC ATGCAGCAGTTGAATAGCTTGGATGCAGAGATGCTGCAGAAAAATCATGA TCAGTTCGCTACGGGCAGCGGCCCTCTTCGTGGCAGTATCACTCAATGCCA AGGGCTGATGCAGTTTTGTGGTGGGGAATTGCAAGCTGAGGCCAGTGCCA TCTTAAACACGCCTGTTTGTGGTATTCCCTTCTCGCAGTGGGGAACTATTG GTGGGGCGGCCAGCGCGTACGTCGCCAGTGGCGTTGATCTAACGCAGGCA GCAAATGAGATCAAAGGGCTGGCGCAACAGATGCAGAAATTACTGTCATT GATGTGA-3′ (SEQ ID NO: 6).

IV. CD8 T Cell Clones Derived from Mice that Survive Y. Pestis Infection Recognize a Single 15-Mer Peptide Derived from YopE

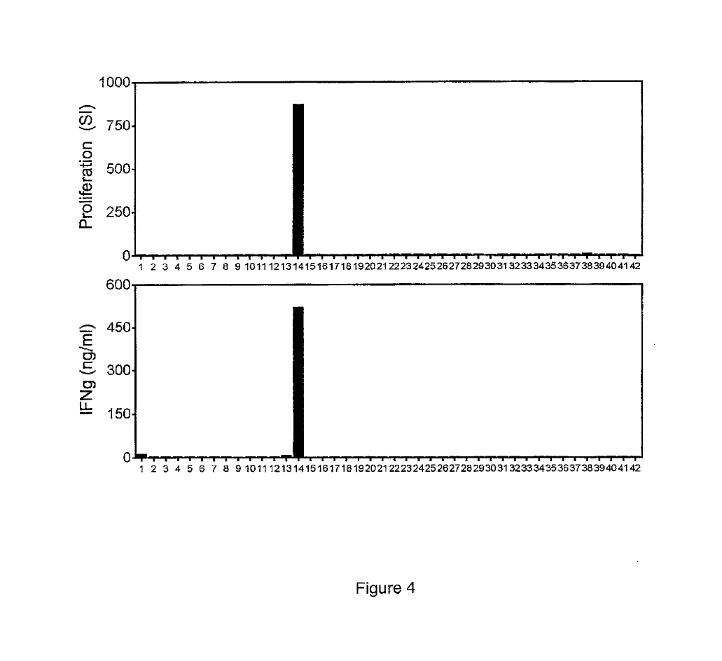

CD8 T cell clones derived from mice that survive Y. pestis infection recognize a single 15-mer peptide derived from YopE (FIG. 4). The specificity of the CD8 clones was assessed further in vitro by re-stimulating the clone with syngeneic mitomycin c-treated splenocytes as antigen presenting cells (APC) and the indicated YopE peptides as antigen. Each of the 42 peptides was assayed at a concentration of 100 nM and consists of a 15-mer that overlaps its neighbors by 10 amino acids. Culture supernatants were harvested 48 hr after initiation of culture and assayed for IFN-γ by ELISA. ³H-thymidine was added 60 hr after the initiation of culture. Incorporation of ³H-thymidine was measured at 72 hr and graphed as fold increase above APC only (Stimulation index, SI). The depicted Vb6+ CD8 clone specifically produced IFN-γ and proliferated in response to YopE peptide number 14. The Vb6-CD8 clone produced an identical response pattern (not shown). The amino acid sequence of peptide number 14 is H₂N-VAHSVIGFIQRMFSE-OH (SEQ ID NO: 1).

The nucleotide sequence encoding the amino acid of SEQ ID NO: 1 is 5′-GTGGCCCACTCTGTGATTGGGTTTATCCAACGCATGTTCTCGGAG-3′ (SEQ ID NO: 2).

V. Immunizing Mice with Attenuated Y. Pestis Primes Recruitment of CD8 T Cells to the Lung that can be Activated by a YopE Peptide

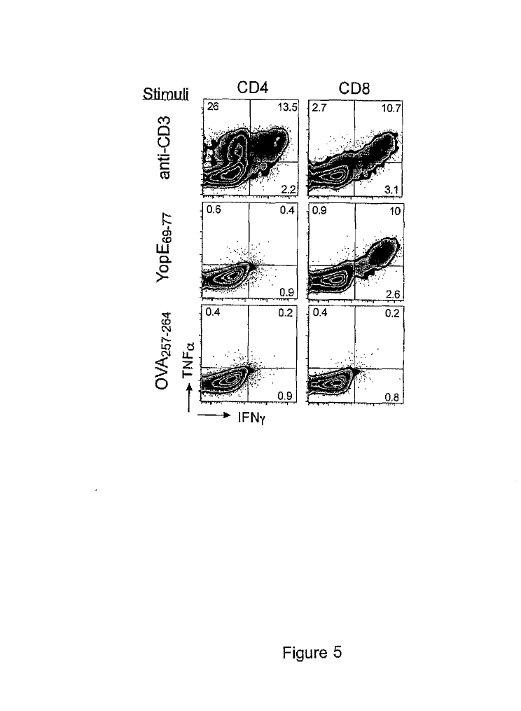

Immunizing mice with attenuated Y. pestis primes recruitment of CD8 T cells to the lung that can be activated by a YopE peptide (FIG. 5). Wild type C57BL/6 mice were immunized with attenuated Y. pestis strain D27pLpxL, boosted 30 days later with a second immunization with Y. pestis strain D27pLpxL, and then challenged 60 days later with virulent Y. pestis strain D27. Four days after challenge, cells were isolated from lungs and cultured in the presence of anti-CD3 monoclonal antibody, a control peptide derived from ovalbumin (OVA), or a 9 amino acid YopE peptide. The amino sequence of this peptide is H₂N-SVIGFIQRM-OH (SEQ ID NO: 3). After 5 hr of culture, cells were harvested and processed for intracellular cytokine staining. Representative flow cytometric plots gated on CD4 or CD8 cells are shown. Results demonstrate that the YopE peptide stimulated TNF-α and IFN-γ expression from nearly as many CD8 cells as did the polyclonal stimulus anti-CD3. The nucleotide sequence encoding the amino acid of SEQ ID NO: 3 is 5′-TCTGTGATTGGGTTTATCCAACGCATG-3′ (SEQ ID NO: 4).

VI. Immunizing Mice with Dendritic Cells Pulsed with a Single YopE Peptide Confers Significant Protection Against Pulmonary Y. Pestis Challenge.

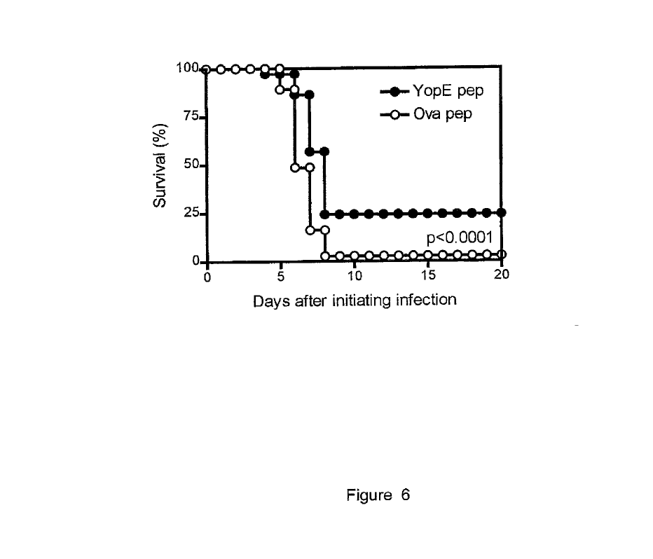

In support of the results described in FIG. 5 above, mice immunized with DCs pulsed with a single YopE peptide were shown to exhibit significantly improved survival after Y. pestis challenge, as compared with control mice immunized with DCs pulsed with an ovalbumin-derived peptide (FIG. 6). Bone marrow cells were cultured with GM-CSF for 8 days to generate DCs. After pulsing for 24 hours with 1 uM YopE peptide H₂N-SVIGFIQRM-OH (SEQ ID NO: 3) or control ovalbumin (OVA) peptide H₂N-SIINFEKL-OH, the DCs were harvested and 5×10⁵ cells were injected intravenously into wild type C57BL/6 mice. After 14 days, the mice were challenged intranasally with 2×10⁵ colony forming units of Y. pestis strain D27. Survival was monitored for 20 days. Data represents the pooled values from 3 individual experiments yielding qualitatively similar results. Immunization with the YopE peptide conferred significantly improved survival (p<0.0001 by log rank test; n=37 mice per group).

VII. Immunizing Mice with a Single YopE Peptide in Adjuvant Confers Significant Protection Against Pulmonary Y. Pestis Challenge.

Intranasal immunization with peptide YopE peptide confers protection against lethal pulmonary Y. pestis challenge (FIG. 6). On days 0, 7 and 21, wild type C57BL/6 mice were immunized intranasally with YopE peptide H₂N-SVIGFIQRM-OH (SEQ ID NO: 3) (10 ug) mixed with cholera toxin (CT) adjuvant (1 ug). Control mice received only CT, or were left untreated (naïve). On day 37, all mice were challenged intranasally with virulent Y. pestis strain D27. In comparison with mice immunized with CT alone, mice immunized with CT and YopE peptide displayed significantly increased survival (p=0.0001 by log rank test; n=10 mice for CT and CT+YopE groups; n=5 for naïve group).

VIII. Mice Immunized with Live Attenuated Y. Pestis Harbor Increased Percentages of CD8 T Cells that Specifically Stain with a MHC K^{b} Tetramer Loaded with a YopE Peptide.

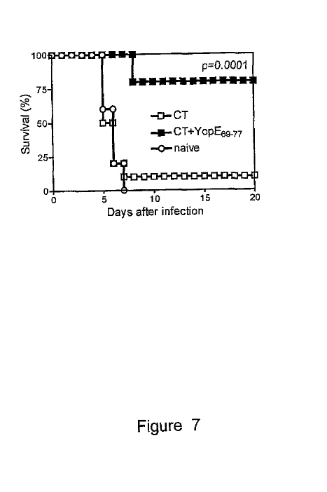

In support of the potential use of YopE to measure CD8 T cell responses to plague vaccines, results demonstrate that mice immunized with live attenuated Y. pestis harbor increased percentages of CD8 T cells that specifically stain with a MHC K^{b} tetramer loaded with a YopE peptide (FIG. 7). The increased frequency of these cells is apparent in both the lung and the peripheral blood (FIG. 7). Upon challenge of the immunized mice with virulent Y. pestis, the frequency of these cells is further increased in both the lung and peripheral blood (FIG. 7). C57BL/6 mice were immunized and boosted with attenuated Y. pestis strain D27-pLpxL and then challenged intranasally with virulent Y. pestis strain D27 as in FIG. 5. At day 4 after challenge, cells were harvested, stained, and analyzed by flow cytometry. Lung cells, peripheral blood leukocytes (PBL) and splenocytes were stained immediately with antibodies to CD4 and CD8 and an MHC class 1 Kb tetramer loaded with the 9 amino acid YopE peptide H₂N-SVIGFIQRM-OH (SEQ ID NO: 3). Plots are representative of five individual mice for each condition. The percentages of lymphocytes staining positive for CD8 and tetramer are shown, with parentheses depicting the percentage of tetramer-positive cells within the CD8 T cell population. YopE tetramer-positive CD8 T cells were detectable in the lungs, blood and spleens of vaccinated mice and their frequency increased dramatically in the lung after Y. pestis challenge.

### IX. Vaccines and Related Assays

Prior studies discounted the utility of YopE as a vaccine component because immunization with YopE failed to induce protective responses in mice (e.g. Leary et al., Microb. Pathog. 26:159-69, 1999, and Ivanov et al., Infect. Immun.76:5181-90, 2008). However, the vaccine formulations used previously for YopE immunizations were best suited to inducing antibody responses. Presumably YopE-specific antibodies responses fail to protect against plague because the T3SS translocates the toxin in a manner that prevents antibody-mediated neutralization of YopE function. Three references (Benner, Leary, Ivanov) have evaluated YopE as a candidate vaccine antigen for Y. pestis and concluded that it was not useful. However, these papers did not evaluate the capacity of YopE to stimulate CD8 T cells. Some researchers are pursuing an attenuated Yersinia (not Y. pestis) strains as vaccine platforms (e.g. Russmann, Wiedig), including the use of YopE fusion proteins to stimulate CD8 responses to the fusion partner rather than YopE itself Stambach et al. demonstrates that YopH can be a target for Y. enterocolitica in mice and suggests YopE might also serve as such a target. However, this paper does not actually demonstrate any such role for YopE, nor advocate the use of YopH or YopE as a vaccine for Y. pestis. Falgarone et al. employs various mutant strains to demonstrate that YopE may be a target for rat CD8 T cells responding to Y. pseudotuberculosis. However, this paper lacks direct evidence that YopE is the target, and does not propose that YopE may be a target for Y. pestis. The authors also do not show that YopE is a protective antigen, nor do they propose using YopE as a vaccine component.

Previous results by the present research group in the plague vaccine field demonstrated that CD8 T cells, TNF-α and IFN-γ contribute to immune defense against pulmonary Y. pestis infection. The present results decisively demonstrate that YopE can be recognized by a large frequency of plague-specific T cells, a finding that is both novel and remarkable. While prior research suggested that YopE might be an antigen recognized by rat CD8 T cells capable of killing cells that were sensitized by exposure to Y. pseudotuberculosis (Falgarone et al., J. Immunol. 162:2875-83, 1999), this suggestion was never demonstrated explicitly nor extended to other Yersinia species. Importantly, the sensitization of cells required that Y. pseudotuberculosis express “invasin”, a protein that is not produced by Y. pestis (see Infection and Immunity, 68:4523-30, 2000). As recently as 2010, leaders in the Yersinia immunology field, including Russman, Wolf-Watz, and Geginat, reported that the Yersinia pseudotuberculosis antigens that are presented to CD8 T cells in a MHC class I context are unknown (Infection and Immunity, Alternative Endogenous Protein Processing via an Autophagy-Dependent Pathway Compensates for Yersinia-Mediated Inhibition of Endosomal MHC II Antigen Presentation, published online ahead of print on 27 Sep. 2010).

Interestingly, several studies have used the amino-terminal portion of Yersinia YopE as a fusion partner for recombinant proteins, in some cases with the goal of inducing robust CD8 T cell responses against the partner of the YopE fusion protein. However, in no instance has it been directly demonstrated that YopE is an antigen recognized by T cells responding to Yersinia infection. Moreover, a recent report screened 101 proteins from Y. pestis, including YopE, for the capacity to activate T cells in mice immunized with attenuated Y. pestis; this report did not identify YopE as an antigen, perhaps because the assay employed likely favored CD4 T cell responses (Li et al., Inf. Immun. 77:4356, 2009). The present data demonstrate that YopE is a dominant CD8 T cell antigen that may serve as a component of Yersinia vaccines, including vaccines for pneumonic plague. In one embodiment, YopE may serve as a useful component of plague vaccines. In other embodiments, plague vaccines comprising YopE, subparts of YopE, genes encoding YopE or its subparts; mixtures of YopE or its subparts with other antigens; or live agents expressing YopE or its subparts are contemplated. In one embodiment, the YopE subpart is the amino acid of SEQ ID NO: 1. In another embodiment, the YopE subpart is the amino acid of SEQ ID NO: 3. In other embodiments the YopE peptide antigen comprises the amino acid sequence of SEQ ID NO: 5 or subparts thereof. In another embodiment, the gene encoding YopE or its subparts is the nucleotide sequence of SEQ ID NO: 2. In yet another embodiment, the gene encoding YopE or its subparts is the nucleotide sequence of SEQ ID NO: 4. In other embodiments the nucleic acid sequence comprises the nucleotide sequence of SEQ ID NO: 6.

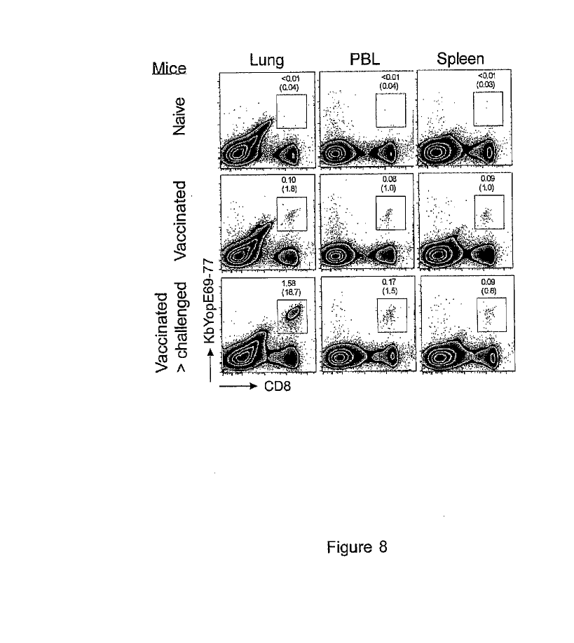

In further embodiments, YopE may serve as a useful component of assays measuring the CD8 T cell responses to plague vaccines, whether those responses are induced by immunization with YopE, subparts of YopE, genes encoding YopE or its subparts; mixtures of YopE or its subparts with other antigens; or live agents expressing YopE or its subparts. In some embodiments, YopE peptides may serve as components in ELISA, ELISpot, and MHC multimer based assays. In other embodiments, the peptides reported in FIGS. 4, 5 and 8 may be used to measure the responses of YopE-specific CD8 T cell in mice expressing the K^{b} MHC molecule. In further embodiments, these and/or other YopE peptides may be useful for other animals, including humans, expressing distinct MHC molecules.

### X. MHC Multimer Based Assays

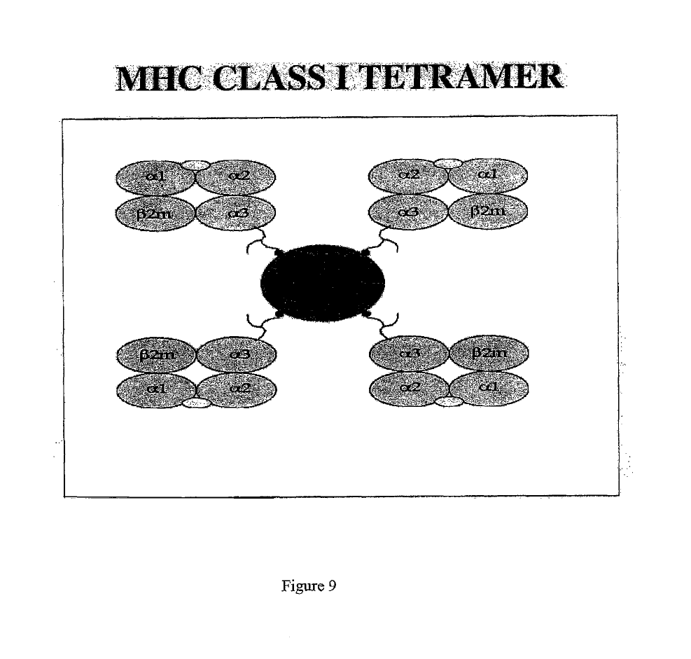

The development of new technologies, tools, and reagents has historically led to great advances in the understanding of immune responses to infection. Recently, a major advance has been the production of soluble major histocompatibility complex class I/peptide tetramers for the direct identification of antigen-specific CD8 T cells by flow cytometry (Altman, J. D., Phenotypic analysis of antigen-specific T lymphocytes. Science 274:94-6 (1996); Busch, D. H., Coordinate regulation of complex T cell populations responding to bacterial infection. Immunity 8:353-62 (1998)). As depicted in FIG. 9, the red oval represents fluorescently labeled streptavidin, which is bound to four (4) biotinylated MHC-I molecules. Each class 1 molecule is a tripartite complex of a heavy chain (alpha1, alpha2, and alpha3 domains), light chain (beta-2-microglobulin/“b2 m”) and peptide antigen (yellow). The MHC-I/peptide complex binds to the T cell receptors on CD8 T cells that are specific for that class I MHC-I/peptide. The tetramer configuration provides stable binding by providing four (4) binding sites, thus increasing avidity. Once bound, the tetramer can be detected using flow cytometry. This methodology allows for the rapid quantification of antigen-specific T cells.

In one embodiment, the MHC multimer based assay may be used to determine the effectiveness of a plague vaccine by measuring the CD8 T cell responses elicited by that vaccine. In a preferred embodiment such an assay may include isolating cells (such as immune cells that include CD8 T cells) from an immunized subject, mixing the cells with the MHC multimer assay reagents in vitro, washing the cells and then determining the number of positively labeled cells using flow cytometry.

1. A vaccine formulation comprising the Y. pestis YopE peptide antigen or subparts thereof.

2. The formulation of claim 1, wherein said YopE peptide antigen comprises the amino acid sequence of SEQ ID NO: 1.

3. The formulation of claim 1, wherein said subpart of said YopE peptide antigen comprises the amino acid sequence of SEQ ID NO: 3.

4. (canceled)

5. A vaccine formulation comprising a nucleic acid sequence encoding the Y. pestis YopE peptide antigen or subparts thereof.

6. The formulation of claim 5, wherein said nucleic acid sequence is in a vector.

7. The formulation of claim 5, wherein said nucleic acid sequence comprises the nucleotide sequence of SEQ ID NO: 2.

8. The formulation of claim 5, wherein said nucleic acid sequence comprises the nucleotide sequence of SEQ ID NO: 4.

9-10. (canceled)

11. A method of vaccination, comprising:
a) providing:
i) a subject at risk of being infected by Y. pestis;
ii) a Y. pestis YopE peptide antigen or subpart thereof; and
b) administering said peptide or subpart thereof to said subject.

12. (canceled)

13. The method of claim 11, wherein said YopE peptide antigen comprises the amino acid sequence of SEQ ID NO: 1.

14. The method of claim 11, wherein said YopE peptide antigen subpart comprises the amino acid sequence of SEQ ID NO: 3.

15-19. (canceled)

20. The method of claim 11, wherein said YopE peptide is expressed by a vector.

21-32. (canceled)

33. An assay, comprising:
a) providing:
i) a cell, and
ii) a MHC-I tetramer/YopE peptide complex;
b) reacting said MHC-I tetramer/peptide complex with said cell under conditions that permit a receptor on said cell to bind to said MHC-I tetramer/peptide complex; and
c) detecting said cell bound to said complex.

34. The assay of claim 33, wherein said cell is an immune cell.

35. The assay of claim 34, wherein said immune cell is a T cell.

36. The assay of claim 34, wherein said T cell is a CD8 T cell.

37. The assay of claim 33, wherein said receptor is a T cell receptor.

38. The assay of claim 33, wherein said cell can respond to Y. pestis.

39. The assay of claim 33, wherein said cell is from a subject infected with Y. pestis.

40. The assay of claim 33, wherein said cell is from a subject immunized with a vaccine against Y. pestis.

41. The assay of claim 33, wherein said peptide is a Y. pestis YopE peptide or subpart thereof.

42. The assay of claim 41, wherein said YopE peptide comprises the amino acid sequence of SEQ ID NO: 1.

43. The assay of claim 41, wherein said YopE peptide comprises the amino acid sequence of SEQ ID NO: 3.

44-45. (canceled)
